(19) United States
(12) Patent Application Publication
(10) Pub. No.: US 2009/0110673 A1
(43) Pub. Date: Apr. 30, 2009

(54) METHODS OF REDUCING CELL DEATH FOLLOWING HYPOXIA / REOXYGENATION

(75) Inventors: Terry L. Vanden Hoek, Chicago, IL (US); Zuo-Hui Shao, Chicago, IL (US); Chang-Qing Li, Chicago, IL (US); David G. Beiser, Chicago, IL (US); Lance Becker, Philadelphia, PA (US)

Correspondence Address: MICHAEL BEST & FRIEDRICH LLP, ONE SOUTH PINCKNEY STREET, P O BOX 1806, MADISON, WI, 53701, US

(73) Assignee: THE UNIVERSITY OF CHICAGO, Chicago, IL (US)

(21) Appl. No.: 12/302,140

(22) PCT Filed: May 25, 2007
(86) PCT No.: PCT/US2007/012535

§ 371 Date: Nov. 24, 2008

Related U.S. Application Data
(60) Provisional application No. 60/803,199, filed on May 25, 2006.

(51) Int. Cl. A61K 31/7084; C12N 5/06; C12N 9/99; C12Q 1/02; A61K 31/27; A01N 1/02; A61K 31/352; A61K 31/192

(52) U.S. Cl. 424/94.1; 435/375; 435/184; 435/29; 435/1.1; 514/456; 514/569; 514/476

(57) ABSTRACT

Provided are methods of reducing cell death, attenuating a burst of reactive oxygen species, reducing cytotoxicity, reducing intracellular oxidant stress due species in a population of cells following hypoxia by reoxygenating the cells in the presence of a reversible electron transport chain inhibitor or under hypercarbic conditions. Also provided is a method to determine the effectiveness of a reversible electron transport chain inhibitor for reducing cell death in a population of cells.

### CROSS-REFERENCE TO RELATED APPLICATIONS

This application claims priority to U.S. Provisional Application No. 60/803,199 filed on May 25, 2006, which is incorporated by reference in its entirety.

### STATEMENT REGARDING FEDERALLY SPONSORED RESEARCH

This invention was made with United States government support under Grant Number 5 R01 HL068951 awarded by the National Institutes of Health/National Heart Lung and Blood Institute. The United States government has certain rights in this invention.

### INTRODUCTION

Reoxygenation of cells following hypoxia has been associated with increased oxidant stress that significantly contributes to tissue injury in several models. Recent work in our cell model suggests that a burst of reactive oxygen species (ROS) is linked to subsequent cell death. Recently, it has been reported that rapid oxidant generation and corresponding tissue injury follow cardiac arrest and CPR in a porcine model. Antioxidants given only at reoxygenation improve cell viability and enhance the return of cell activity and tissue function. Interestingly, classical ischemic preconditioning protection against ischemia/reperfusion (I/R) injury in a cardiomyocyte model is associated with significant attenuation of reperfusion oxidants.

Injury following transient hypoxia may result from the direct injury occurring during the hypoxic interval and from the indirect injury caused by reoxygenation, which can be more severe than the direct injury. Reoxygenation injury is relevant to many fields of medicine including cardiology, transplant surgery, plastic surgery, orthopedic surgery, and emergency medicine.

### SUMMARY

In one aspect, the present invention provides a method of reducing cell death in a population of cells following hypoxia comprising reoxygenating the cells in the presence of an effective amount of a reversible electron transport chain inhibitor.

In another aspect, the present invention provides a method of reducing cell death in a population of cells following hypoxia within a subject comprising reoxygenating the cells in the presence of an effective amount of a reversible electron transport chain inhibitor.

In another aspect, the present invention provides a method of attenuating a burst of reactive oxygen species in a population of cells following hypoxia comprising reoxygenating the cells in the presence of an effective amount of a reversible electron transport chain inhibitor.

The invention also provides a method of reducing cytotoxicity in a population of cells following hypoxia comprising reoxygenating the cells in the presence of an effective amount of a reversible electron transport chain inhibitor.

In addition, the present invention provides a method of reducing intracellular oxidant stress in a population of cells following hypoxia comprising reoxygenating the cells in the presence of an effective amount of a reversible electron transport chain inhibitor.

The present invention further provides a method of preserving a harvested organ or tissue comprising reoxygenating the organ or tissue in the presence of an effective amount of a reversible electron transport chain inhibitor.

Further, the present invention provides a method of determining the effectiveness of a reversible electron transport chain inhibitor for reducing cell death in a population of cells following hypoxia comprising reoxygenating the cells in the presence of a reversible electron transport chain inhibitor and assessing the effect on cell death.

In another aspect, the present invention provides a method of reducing cell death in a population of cells following hypoxia comprising reoxygenating the cells with oxygen under a hypercarbic condition.

In another aspect, the present invention provides a method of attenuating a burst of reactive oxygen species in a population of cells following hypoxia comprising reoxygenating the cells with oxygen under a hypercarbic condition.

The invention also provides a method of reducing cytotoxicity in a population of cells following hypoxia comprising reoxygenating the cells with oxygen under a hypercarbic condition.

In addition, the present invention provides a method of reducing intracellular oxidant stress in a population of cells following hypoxia comprising reoxygenating the cells with oxygen under a hypercarbic condition.

The present invention further provides a method of preserving a harvested organ or tissue comprising reoxygenating the organ or tissue with oxygen under a hypercarbic condition.

### BRIEF DESCRIPTION OF THE DRAWINGS

FIG. 1A shows attenuation of DCF fluorescence following contacting cells with diethyldithiocarbamic acid (“DDC”) (1 mM) during reperfusion from a control peak of maximal intensity of 2.8±0.2 a.u. to 1.6±0.1 a.u. (n=5).

FIG. 1B shows reduction of I/R induced cell death following contacting cells with DDC (1 mM) during reperfusion from a control of 49.7±6.7% to 15.7±4.7% (n=5, p<0.01).

FIG. 2A shows attenuation of DCF fluorescence following contacting cells with 2-anthracene-carboxylic acid (0.1 μM) for the first 10 minutes of reperfusion from a control peak of 2.8±0.2 a.u. to 1.7±0.1 a.u. in treated cells (n=3, p<0.05).

FIG. 2B shows reduction of I/R induced cell death following contacting cells with 2-anthracene-carboxylic acid (0.1 μM) for the first 10 minutes of reperfusion from a control percent death of 47.1±3.0% (n=7) to 26.1±4.0% (n=5, p<0.01).

FIG. 3 shows reduction of I/R cell death following contacting cells with exogenous α-NADH (20 μM) from a control percent death of 47.1±3.0% to 13.8±1.3% (n=5, p<0.01). Cells treated with exogenous β-NADH (20 μM) showed no benefit. The inset shows that cells treated with exogenous β-NADH (20 μM) showed a percent increase in ROS generation during reperfusion over ischemia from 6.2±19.6% in untreated cells to 511±123% (n=8, p<0.01). When this same group was co-treated with α-NADH (20 μM), reversal of the increase was observed.

FIG. 4A shows attenuation of maximal peak DCF fluorescence intensity following contacting cells with stigmatellin (2-20 nM) during the first 15 min of reperfusion from a control of 2.4±0.3 a.u. to 0.46±0.02 a.u. (n=5, p<0.001).

FIG. 4B shows reduction of I/R cell death following contacting cells with stigmatellin (2-20 nM) for the first 15 min of reperfusion from a control of 53.8±3.5% to 10.8±2.9% (n=5, p<0.001). The inset shows different doses and durations of stigmatellin and the impact on cell death from a control of 50.2±3.0% to increased cell death with a 3 hour 100 μM treatment of 80.1±6.0% (n=3, p<0.01) to a 15 minute 100 μM treatment of 52.3±3.0% (n=3) to a decreased cell death with a 15 minute 20 nM treatment (when compared to the same controls) of 20.3±5.0% (n=3, p<0.001).

FIG. 5A shows increased cell death with treatment with rotenone at 10 μM, 1 μM and 100 nM during the first 15 minutes of reperfusion from a control of 50.2±3.0% to 66.2±3.4% (n=3, p<0.01), 77.1±6.6% (n=3, p<0.01) and 61.3±1.6% (n=3, p<0.01), respectively.

FIG. 5B shows that rotenone is readily able to attenuate reperfusion ROS (5 μM) from a maximal peak fluorescence intensity of 2.4±0.3 a.u. to 0.7=0.1 a.u. (n=5, p<0.01).

FIG. 6 highlights the location of mitochondrial inhibitors used in this study and the relation of the dismutation of superoxide to hydrogen peroxide in the inter-membrane space or cytosol.

FIG. 7 shows increase in I/R cell death with hypocarbic reperfusion conditions (pCO₂ 7 torr, pH 7.9, n=5) as compared to normocarbic reperfusion conditions (PCO₂ 36 torr, pH 7.4, n=15) and a decrease in I/R cell death with hypercarbic reperfusion conditions (pCO₂ 71 torr, pH 6.8, n=15) as compared with normocarbic conditions (analysis of variance, group main effect p<0.001; group-by-time interaction p<0.001). *p<0.05; #p<0.01; & p<0.001.

FIG. 8A shows the increase of dichlorofluorescin (DCF) fluorescence (indicator of ROS generation) maximal intensity within the first 10 minutes of reperfusion under hypocarbic conditions (two-fold increase) in comparison to normocarbic conditions (n=4) and the attenuation of DCF under hypercarbic conditions (n=4) (analysis of variance, group main effect p=0.045; group-by-time interaction p=0.006).

FIG. 8B shows the ability of treatment with L-NAME to reverse the hypercarbic condition-induced attenuation of DCF (n=5) (analysis of variance, group main effect p<0.068; group-by-time interaction p=0.37). *p<0.05.

FIG. 9A shows a spike in nitric oxide (NO) generation indicated by 4,5-diaminofluorescin (DAF-2) fluorescence early in reperfusion by hypocarbic reperfusion conditions as compared with control, which declined gradually to similar levels of the normocarbic conditions (analysis of variance, group main effect p=0.076; group-by-time interaction p=0.002).

FIG. 9B shows no spike in NO generation during the first 15 minutes of reperfusion under hypercarbic conditions but a sustained generation of NO leading to higher levels of NO thereafter in late reperfusion as compared with normocarbic conditions (n=8) (analysis of variance, group main effect p=0.13; group-by-time interaction p=0.0008). *p<0.05 ; #p<0.01. I/R, ischemia/reperfusion; a.u., arbitrary units.

FIG. 10A shows the reduction of the sustained increase of NO generation by measuring DAF-2 fluorescence induced by hypercarbic conditions back to control levels by L-NAME (n=8) (analysis of variance, group main effect p=0.24; group-by-time interaction p=0.014).

FIG. 10B shows L-NAME reduces the protective effect of hypercarbic reperfusion (increased cell death) (n=3) (analysis of variance, group main effect p=0.052; group-by-time interaction p=0.032) where the normocarbic cell death reference was provided as a dotted line tracing. *p<0.05.

FIG. 11A shows administration of stigmatellin (STG) (20 nM) during the first 15 minutes of reperfusion attenuates ROS burst (DCF fluorescence) caused by hypocarbic conditions (n=5) (analysis of variance, group main effect p=0.069; group-by-time interaction p=0.02).

FIG. 11B shows administration of apocynin (300 μM) given during reperfusion increased the ROS burst (DCF fluorescence) in hypocarbic conditions (analysis of variance, group main effect p=0.002; group-by-time interaction p=0.023).

FIG. 11C shows administration of apocynin (300 μM) given during reperfusion increased the ROS burst (DCF fluorescence) in normocarbic reperfusion (analysis of variance, group main effect p=0.011; group-by-time interaction p=0.012).

FIG. 11D shows stigmatellin administered during the first 15 minutes of reperfusion reduced cell death caused by hypocarbic reperfusion (n=5) (analysis of variance, group main effect p=0.0078; group-by-time interaction p=0.0005). *p<0.05; #p<0.01; & p<0.001.

### DETAILED DESCRIPTION

Post resuscitation injury can be reduced by minimizing reperfusion injury during reoxygenation of tissues after ischemia. We have found that reoxygenation in the presence of an electron transport chain inhibitor or reoxygenation under hypercarbic conditions reduces post-resuscitation injury, in part by modifying mitochondrial oxidants or nitric oxide synthase-induced nitric oxide production.

Mitochondria are necessary for maintenance of cellular metabolism and play a unique role as a potent source of oxidants that can both elicit protective stress responses and can, when critical oxidant stress thresholds are surpassed, initiate apoptosis and irreversible damage. It has previously been reported that mitochondrial superoxide generation increases during ischemia prior to reperfusion. A burst of reactive oxygen species (ROS) within minutes of reoxygenating ischemic cardiomyocytes is associated with cytochrome-c release, initiation of the apoptotic cascade and eventual cellular demise.

We postulated that the reintroduction of oxygen into a highly reduced cellular redox environment rapidly stimulates an otherwise dormant ETC, resulting in a transient, robust burst of ROS. The examples tested whether the ROS burst seen at reperfusion also originates from mitochondria.

The examples below demonstrate that partial, reversible inhibition of the mitochondrial electron transport chain in cells following hypoxia during reoxygenation attenuates ROS production in cells, enhances recovery of cell activity and tissue function, reduces cytotoxicity, increases cell viability, and reduces extracellular oxidant stress. Results of the examples below also show that CO₂ and pH changes induced during the reoxygenation can significantly modify post-resuscitation oxidants and injury. Methods of modulating CO₂ levels during reperfusion enhance recovery of cell activity and tissue function and increase cell viability after ischemic injury.

An “electron transport chain inhibitor” is an inhibitor of any stage of mitochondrial electron transport. Four membrane-bound protein complexes are part of the mitochondrial ETC: Complex I (NADH dehydrogenase); Complex II (succinate dehydrogenase); Complex III (cytochrome bc₁ complex); and Complex IV (cytochrome c oxidase). Electron transfer between these complexes is accomplished by the mobile coenzymes.

Suitably, the electron transport chain inhibitor has the ability to rapidly attenuate the ROS burst detected at reoxygenation, but does not interfere with return of cell activity necessary to support tissue function. Though ATP was not measured directly in the chick cardiomyocyte cell model, the return of spontaneous contractions is a reliable indicator of functional mitochondrial recovery and ATP production. Competitive or reversible inhibitors of the ETC were shown to be most effective.

During the critical transition from hypoxia into reoxygenation, mitochondrial superoxide production and subsequent dismutation to H₂O₂ is largely responsible for the reoxygenation ROS burst. In addition, NADH-oxidases are linked to the generation of reoxygenation oxidants, such as ROS, and injury. By manipulating the pool of reducing equivalents available for substrate utilization (via exogenous addition of α-NADH and β-NADH), the examples herein establish that the electron transport chain is involved in producing the ROS burst.

The biological properties of α-NADH render it suitable for reducing reoxygenation injury. α-NADH is physiologically inert, and competes with the β-isoform as a potential reducing equivalent substrate for the ETC during reoxygenation. α-NADH is not easily imported into the mitochondria or oxidized by Complex I and, therefore provides no reducing equivalents for the ETC. As a result, α-NADH partially inhibits the ETC. This unique approach may offer a new means of a ‘controlled metabolic reperfusion’ which targets the upstream events of the respiratory chain by modulating ETC substrates.

As demonstrated in the examples below, 2-anthracene-carboxylic acid (“Rhein tech”) and α-NADH are suitable for use in reducing ROS and providing functional protection. As competitive inhibitors of Complex I, 2-anthracene-carboxylic acid and α-NADH partially and reversibly inhibit the electron transport chain. More importantly, the dose of 2-anthracene-carboxylic acid administered to achieve protection was shown to decrease mitochondrial respiration by only 10-15%.

The examples below show that stigmatellin attenuates reperfusion ROS and improves cell viability. Stigmatellin is a reversible inhibitor of the quinol oxidation site (Qₒ) of cytochrome bc1 of Complex III (ubiquinol: ferricytochrome c oxidoreductase). Stigmatellin inhibits electron flow through the Qₒ site of Complex III. The Qₒ site can be a significant source of superoxide production if electron flow through this site is bypassed. Bypassing this site is believed to be responsible for producing superoxide in the inter-mitochondrial membrane space/cytosol, where superoxide can be rapidly converted to other more damaging oxidants like H₂O₂ and OH⁻ (via Fenton chemistry). Stigmatellin binds to the Rieske iron sulfur proteins (ISP) in the distal niche of Qₒ site and can effectively block electron transfer to cytochrome c1, preventing the bypass reaction responsible for this oxidant generation. Developing a protocol that achieved successful treatment of post-resuscitation injury with stigmatellin proved more difficult than expected. When stigmatellin was given during the whole 3 hour course of reperfusion, ROS was attenuated but the cardiomyocytes still demonstrated significant cell death, similar to the observed rotenone phenomenon. The binding affinity of stigmatellin to Complex III, however, is reversible. Thus, administration of stigmatellin for only the first few minutes of reperfusion was attempted. Unfortunately, using a high dose of stigmatellin (100 μM) for only the first 15 min of reperfusion, ROS was abrogated but the increase in cell viability was less than that seen for 2-20 nM stigmatellin. However, when administering nanomolar range doses (2-20 nM) of stigmatellin for only the first 15 minutes of reperfusion, both ROS attenuation and cardioprotection were achieved.

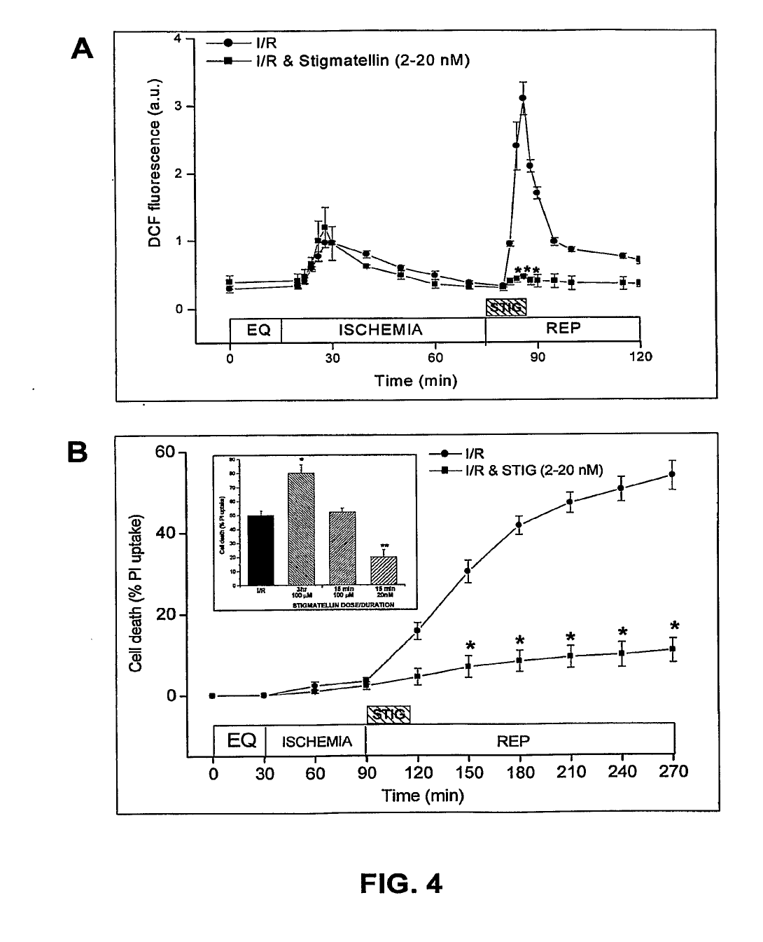

As seen in the inset of FIG. 4B, applying the correct dose and timing of stigmatellin is critical in obtaining cardioprotection. Cells treated with 100 μM stigmatellin for 3 hours of reperfusion significantly increased cell death over a control of 50.2±3.0 % to 80.1±6.0% (n=3, p<0.01), and even when the duration of exposure was shortened to the shortest treatment window tested (first 15 minutes of reperfusion), cell death remained unchanged at 52.3±3.0 % (n=3). However, when doses of stigmatellin were lowered to 20 nM, and the same 15 minute course of administration used, cell death was significantly attenuated from the control of 50.2±3.0 % to 20.3±5.0% (n=3, p<0.01). One of ordinary skill in the art would appreciate that other doses of stigmatellin may be suitable for use in the methods of the present invention.

The results presented in the examples below indicate that transient inhibition of the Qₒ site of Complex III is a novel and highly specific target for post-resuscitation therapy. Treatment with stigmatellin highlights the importance of developing a paradigm of post-resuscitation treatment whereby inhibitor treatment is targeted to attenuate ROS during the early minutes of reoxygenation.

Rotenone, another inhibitor of Complex I, was also tested. In striking contrast to the other tested ETC inhibitors, there was not any dose of rotenone that could confer protection against I/R injury. While rotenone attenuated the reperfusion oxidant burst (confirming that mitochondria are an important source of reperfusion oxidants), no tested dose of rotenone conferred cell protection. Even nanomolar doses administered for only the first 15 minutes of reperfusion did not give cardioprotection despite attenuation of ROS. Unlike 2-anthracene-carboxylic acid and α-NADH, rotenone has been described in several studies as an irreversible Complex I inhibitor. Rotenone can shut down electron transport and attenuated the damaging oxidant stress. However, permanent inhibition of Complex I is deleterious to cell viability. In fact, higher doses of rotenone were actually shown to augment post-resuscitation injury.

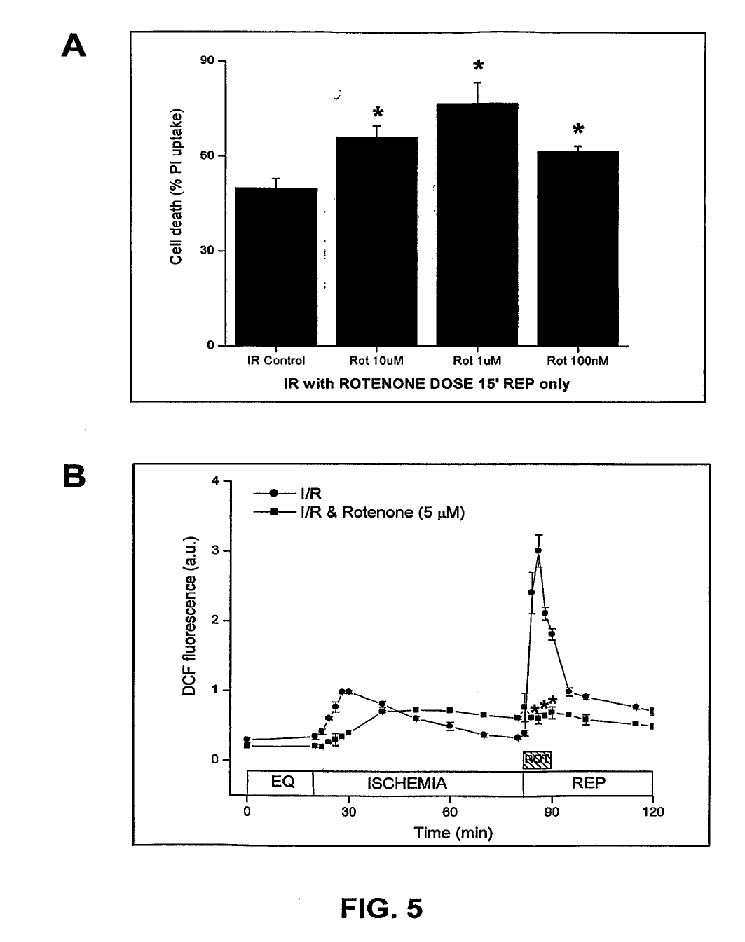

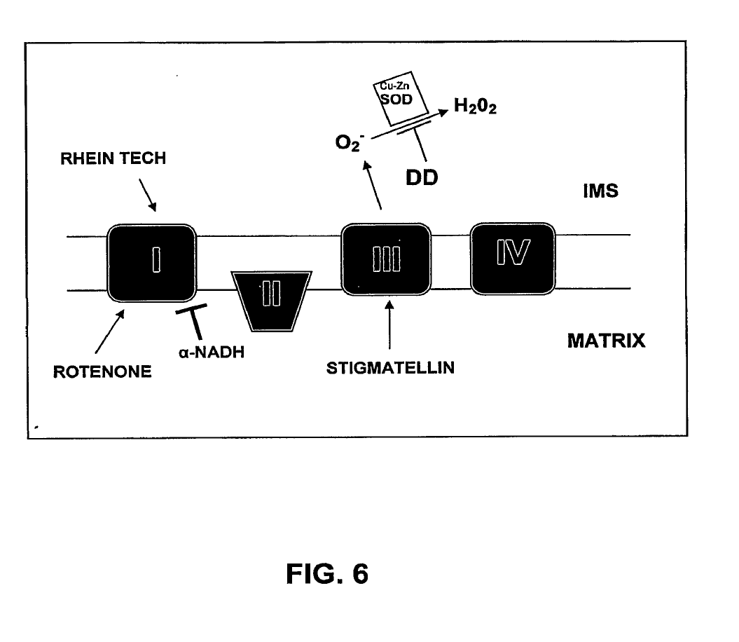

FIG. 5A shows that when both the shortest duration of exposure is selected (first 15 minute reperfusion) and serial dilutions of 10 μM, 1 μM and 100 nM of rotenone are applied cell death increases from a control of 50.2±3.0% to 66.1±3.4% (n=3, p<0.01), 77.4±6.6% (n=3, p<0.01) and 61.3±1.6% (n=3, p<0.01), respectively. Surprisingly, even when nanomolar doses of rotenone (100 nM) are applied cell injury persists. However, as FIG. 5B shows, rotenone is readily able to attenuate reperfusion ROS (dose is 5 μM, but representative of all doses) from a peak fluorescence intensity of 2.4±0.3 a.u. to 0.7±0.1 a.u. (n=5, p<0.01).

In addition to the reversible inhibitors exemplified herein, including DDC, α-NADH, stigmatellin, and 2-anthracene-carboxylic acid, one of ordinary skill in the art would appreciate that other reversible electron transport inhibitors are suitable for use in the methods of the present invention. For example, sodium cyanide, amobarbital, UHDBT, myxothiazol, antimycin and MOA stilbene may be suitable for use in the methods of the present invention.

A reversible inhibitor useful in the present invention suitably has an in vitro dissociation constant, K_{d}, of at least 10⁻², 10⁻³, or 10⁻⁴ M, but not greater than 10⁻⁶, 10⁻⁷, 10⁻⁸, 10⁻⁹, 10⁻¹⁰, or 10⁻¹¹ M. If the reversible inhibitor can bind to the enzyme active site in place of the substrate, it is described as a “competitive inhibitor.”

As would be appreciated by one of ordinary skill in the art, suitable reduction in cell death and protection from reoxygenation can be obtained by varying the doses, durations of exposure and/or degrees of reversibility of the electron transport chain inhibitors. It is well within the skill of one of ordinary skill in the art, using the teachings provided herein, to determine the effective amount for any given reversible electron transport chain inhibitor.

An effective amount of the reversible electron transport chain inhibitor is an amount sufficient to attenuate production of ROS and improve cell viability. Suitably, an effective amount of the reversible electron transport chain inhibitor is an amount that reduces cell death following hypoxia and reoxygenation by about 30%, about 40%, about 50%, about 60%, about 70%, about 80%, or about 90% relative to an untreated control. An effective amount of a reversible electron transport chain inhibitor is also an amount that attenuates the burst of ROS following hypoxia and reoxygenation by about 30%, about 40%, about 50%, about 60%, about 70%, about 80%, or about 90% relative to an untreated control. Further, an effective amount of a reversible electron transport chain inhibitor is an amount that reduces reperfusion-associated cytotoxicity by about 30%, about 40%, about 50%, about 60%, about 70%, about 80%, or about 90% relative to an untreated control. An effective amount of a reversible electron transport chain inhibitor is an amount that reduces intracellular oxidant stress due to ROS following hypoxia and reoxygenation by about 30%, about 40%, about 50%, about 60%, about 70%, about 80%, or about 90% relative to an untreated control.

Suitably, the cells are contacted with the electron transport chain inhibitor for about the first 5, 10, 15, 20, 25, or 30 minutes of reoxygenation following hypoxia. Suitably, the cells are contacted with the electron transport chain inhibitor as close to the start of reoxygenation as possible. For example, contact could begin at about the same time as reoxygenation or about 5, 10, or 15 minutes after reoxygenation begins.

The reversible electron transport chain inhibitor may be administered in a pharmaceutical formulation. Suitably, the pharmaceutical formulation contains the electron transport chain inhibitor and suitable excipients. The pharmaceutical formulation may be administered to a subject in an amount effective in reducing cell death or formation of ROS. The subject is suitably a mammal, more suitably a human. The pharmaceutical formulation may be administered in any suitable manner, such as, parenterally. The reversible electron transport chain inhibitor may also be part of an organ or tissue preservation solution.

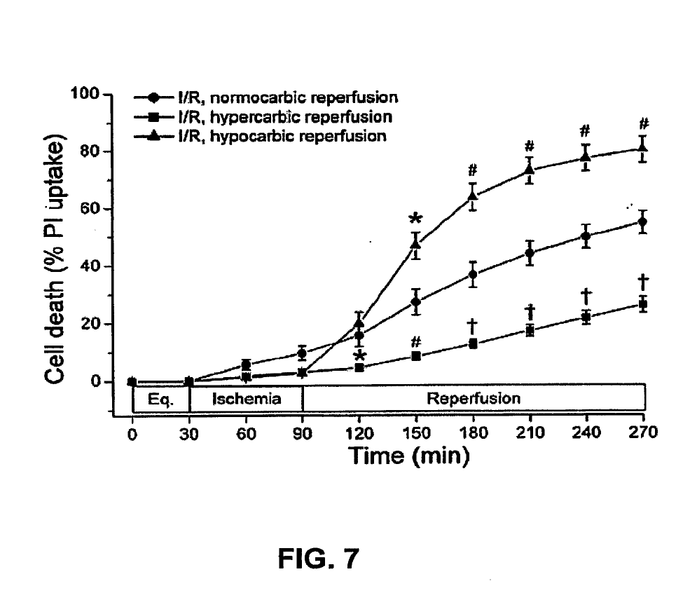

Alternatively, contacting cells in a cell population with oxygen under hypercarbic conditions can reduce reperfusion injury. Results of the examples below demonstrate that contacting cells with oxygen under hypercarbic conditions during reoxygenation after ischemia as compared to normocarbic conditions can reduce cell death, increase recovery and function of tissue, attenuate ROS production, and increase sustained generation of NOS-mediated nitric oxide. The use of hypercarbic conditions within the first 15 minutes of reoxygenation of cells or tissue following hypoxia or ischemia leads to an increased survival of cells (increased viability), an attenuation of the burst of ROS that occurs during reoxygenation, a reduction in reperfusion-associated cytotoxicity, and a reduction in intracellular oxidant stress. As demonstrated in FIG. 7, cell death after ischemia is greatly reduced when reoxygenation is conducted under hypercarbic conditions, as seen by a reduction in cell death from 54.8%±4.0% for normocarbic conditions to 26.3%±2.8% at 270 minutes after the start of reoxygenation.

Hypercarbic conditions for reoxygenation include conditions in which the pCO₂ levels are greater than normocarbic conditions. “Normocarbic condition” is a condition in which the pCO₂ is about 36 torr and the pH is about 6.8. Suitably, a hypercarbic condition has pCO₂ of greater than 36 torr, for example about 40 torr, about 45 torr, about 50 torr, about 55 torr, about 60 torr, about 65 torr, about 70 torr, about 80 torr, about 90 torr or about 100 torr. A hypercarbic condition may be created by any suitable means known to one skilled in the art. For example, administration of oxygen under a hypercarbic condition may be created by co-administration of O₂ and CO₂ in the form of a gas or a solution, e.g. a buffered solution. As would be appreciated by one of ordinary skill in the art, buffer solutions may be suitable for physiological administration to a cell, e.g., a TRIS, sodium carbonate, sodium bicarbonate or a combination thereof, buffered salt solution. Examples of a buffered hypercarbic solution to administer oxygen to a cell are demonstrated in the examples below, but it is contemplated that other methods known in the art may be used to create a hypercarbic solution. The pH may also be controlled. For example, the pH may be less than about 7.4, or less than about 7.0, or less than about 6.8. Also, administration of oxygen under hypercarbic conditions may be combined with the administration of an electron transport inhibitor as described above.

Suitably, an effective hypercarbic condition is one that reduces cell death following hypoxia and reoxygenation by about 30%, about 40%, about 50%, about 60%, about 70%, about 80%, or about 90% relative to an untreated control. An effective hypercarbic concentration is also one that attenuates the burst of ROS following hypoxia and reoxygenation by about 30%, about 40%, about 50%, about 60%, about 70%, about 80%, or about 90% relative to an untreated control. Further, an effective hypercarbic condition is one that reduces reperfusion-associated cytotoxicity by about 30%, about 40%, about 50%, about 60%, about 70%, about 80%, or about 90% relative to an untreated control. An effective hypercarbic condition is one that reduces intracellular oxidant stress due to ROS following hypoxia and reoxygenation by about 30%, about 40%, about 50%, about 60%, about 70%, about 80%, or about 90% relative to an untreated control.

Suitably, the cells are contacted with oxygen under the hypercarbic conditions for about the first 5, 10, 15, 20, 25, or 30 minutes of reoxygenation following hypoxia. The cells are suitably contacted with oxygen under the hypercarbic conditions as close to the start of reoxygenation as possible. For example, contact could begin at about the same time as reoxygenation or about 5, 10, or 15 minutes after reoxygenation begins.

This invention is envisioned to cover optimizing the decline of tissue CO₂ for a number of minutes after return of spontaneous circulation after cardiac arrest, post-resuscitation, or ischemic injury in a subject. It is contemplated that optimized ventilation and controlled reoxygenation strategies under hypercarbic conditions can be used to regulate CO₂ levels to decrease mitochondrial-mediated ROS oxidant damage and increase survival results of a subject. It is envisioned that during initiation of reoxygenation, oxygen will be administered at more hyperbaric conditions, and as reoxygenation progresses, the pCO₂ will be gradually reduced over a period of time until oxygen is administered under normocarbic conditions.

It is also envisioned that regulation of CO₂ levels within a patient by controlling the delivery of oxygen under hypercarbic conditions in conjunction with pharmaceutical compositions comprising the reversible electron chain inhibitor. In some embodiments, a therapeutically effective dose of the electron chain inhibitor used in combination with delivery of oxygen under hypercarbic conditions may be less than the amount that would be therapeutically effective if the electron chain inhibitor was administered alone. As understood by one skilled in the art, the levels of hypercarbic conditions for delivery of oxygen when combined with an electron chain inhibitor may be less hypercarbic than the use of hypercarbic conditions alone, for example, the hypercarbic condition may approximate normocarbic conditions.

The methods of the present invention may also be used to preserve harvested organs or tissues. Harvested organs and tissues may be placed in a buffered solution with the electron transport inhibitor for a given amount of time after harvesting, e.g. 10 minutes, 15 minutes, 20 minutes, or 30 minutes. Harvested organs and tissues may also be placed in a hypercarbic solution in the presence of oxygen. Delivery of oxygen under hypercarbic conditions or addition of an electron transport chain inhibitor may be used in conjunction with other methods of preserving organs or tissues known to one skilled in the art.

Another embodiment of the invention may be a method of determining the effectiveness of a reversible electron transport chain inhibitor for reducing cell death due to reoxygenation following hypoxia. The method includes contacting a cell undergoing reoxygenation following hypoxia with a reversible electron transport chain inhibitor and assessing the effect on cell death. Cell death can be assayed by any suitable method known by one skilled in the art, for example, the quantification of the number of cells stained with propidium iodine with or without treatment with the electron transport chain inhibitor.

For the purposes of this invention, “hypoxia” is defined as a condition in which the body as a whole (generalized hypoxia), a region of the body (tissue hypoxia), or a harvested organ or other tissue is deprived of adequate oxygen supply. Hypoxia in which there is substantially complete deprivation of oxygen supply is referred to as anoxia.

Generalized hypoxia may be caused by low levels of oxygen in the blood (hypoxemia) or by the inability of tissues throughout the body to utilize the oxygen supplied. For example, generalized hypoxia occurs when there is an inadequate supply of oxygen due to low partial pressure of atmospheric oxygen, inadequate pulmonary ventilation (e.g. in chronic obstructive pulmonary disease or respiratory arrest); or shunts in the pulmonary circulation or a right-to-left shunt in the heart. In addition, carbon monoxide poisoning, which inhibits hemoglobin's ability to bind oxygen, can cause generalized hypoxia. Hypemic hypoxia occurs when there is an inability of the blood to carry oxygen and histotoxic hypoxia occurs when the quantity of oxygen reaching the cells is normal, but the cells are unable to effectively use the oxygen. Another example of generalized hypoxia is anemic hypoxia in which arterial oxygen pressure is normal, but total oxygen content of the blood is reduced. This may be due to reduced hemoglobin content in erythrocytes or decreased hematocrit e.g. from blood loss (blood loss anemia).

Tissue hypoxia includes, but is not limited to, ischemic, or stagnant hypoxia in which there is a local restriction in the flow of otherwise well-oxygenated blood (for example cerebral ischemia and ischemic heart disease), cerebral hypoxia in which the brain is deprived of oxygen despite normal blood flow, and intrauterine hypoxia.

The following examples are meant to be illustrative only and are not intended as a limitation on the concepts and principles of the invention.

### EXAMPLE 1

Materials

Stigmatellin, rotenone, diethyldithiocarbamic acid (DDC), NADH isomers were obtained from Sigma (St. Louis). 2-anthracene-carboxylic acid (rhein tech) was purchased from Aldrich (Milwaukee, Wiss.).

Cardiac Cell Culture

Ventricular embryonic chick cardiomyocytes were prepared according to published procedure. (Vanden Hoek T L, Becker L B, Shao Z, et al. Reactive oxygen species released from mitochondria during brief hypoxia induce preconditioning in cardiomyocytes, J. Biol. Chem. 199; 273:18092-18098, which is incorporated herein by reference). 10-day old chicken embryo hearts were removed, ventricular tissue minced into 0.5 mm fragments, enzymatically dispersed with 0.025% trypsin (Life Technologies, New York, N.Y.), centrifuged, 0.7×10⁶ cells were pipetted onto 25 mm coverslips, and incubated with 5% CO₂. Coverslips were checked for non-muscle cell contamination. Experiments were performed with 3-5 day cardiac cell cultures, by which time a synchronously contracting layer of cells could be visualized and viability exceeded 95%.

Perfusion

Coverslips with contracting cells were placed in a 1.2 mL Sykes-Moore perfusion chamber (Bellco Glass Inc., Vineland, N.J.). The chamber and inflow tubing were maintained at 37° C. Perfusate was supplied to the chamber (0.25 ml/min) via stainless steel tubing to minimize diffusive entry of ambient O₂.

Normoxic perfusate used for baseline conditions and for reperfusion subsequent to ischemia contained oxygenated balanced salt solution (BSS) with a PO₂ OF 149 torr, PCO₂ of 40 torr, pH of 7.4, and [K]⁺ of 4.0 mEq/L, and glucose (5.6 mM). To simulate ischemia, the perfusate contained BSS with 2-deoxyglucose (2-DOG) (20 mM) rather than glucose and [K]⁺ of 8.0 mEq/L. The ischemic perfusate was equilibrated with 80% N₂ and 20% CO₂ prior to use to produce a PO₂ of ˜5 torr, a PCO₂ of 144 torr and a final pH of 6.8.

Fluorescence Microscopy

Cells were imaged with an Olympus IMT-2 inverted phase/epifluorescent microscope equipped with Hoffman Modulation optics to accentuate surface topology of the cells. Phase contract Hoffman Modulation optics and a CCD camera were used to monitor contractions and morphologic membrane changes in the same filed of cells (approximately 70×90 μm) over time. Fluorescent images were acquired from a cooled slow-scanning PC-controlled camera (Hamamatsu, Hamamatsu City, Japan), and changes in fluorescence activity over time were quantified with Image-One software (Image-Pro Plus). Fluorescence was standardized periodically using 2.5% uranium microspheres in a metal grid and silicate glass base and mounted on a microscope slide.

Viability Assay

Cell viability was assessed with the fluorochrome propidium iodide (PI) (5 μM, Molecular Probes, Eugene, Oreg.). PI is excluded from viable cells, but enters cells and binds to chromatin after loss of cell membrane integrity. The cells then become highly fluorescent (excitation wavelength of 540 nm and a 590 nm band pass emission filter). PI was used to quantify cell death throughout the entire time of each experiment. PI exhibited minimal toxicity in control cells even after a ten-hour exposure. All cells in the field studied were stained with PI at the end of the experiment by permeabilizing the cells with digitonin (300 μM). Percent cell death was then calculated as the PI fluorescence at any given time point relative to the maximal fluorescence value seen after digitonin exposure.

Cell Contraction

Contractions were assessed by monitoring synchronous movement within the same field of cells as previously reported. A return of contraction following simulated ischemia/reperfusion was indicated when contractions could be seen throughout the field of cells following the three hour period of reperfusion.

Measurement of Intracellular Reactive Oxygen Species (ROS)

Intracellular oxidant stress due to ROS was monitored real time with the intracellular probe 2′,7′-dichlorofluorescin diacetate (DCFH-DA, 5 μM, Molecular Probes, Eugene, Oreg.) as described in published literature (Vanden Hoek, F L, et al., Reactive oxygen species released from mitochondria during brief hypoxia induce preconditioning in cardiomyocytes, J. Biol. Chem. 1988; 273:18092-18098, which is incorporated by reference herein; Vanden Hoek, T L, et al, Preconditioning in cardiomyocytes protects by attenuating oxidant stress at reperfusion, Circ Res 2000; 86:534-540, which is incorporated by reference herein; Vanden Hoek, T L, Reperfusion, not simulated ischemia initiates intrinsic apoptosis injury in chick cardiomyocytes Am J. Physiol Heart Physiol 2003; 284:H141-150, which is incorporated by reference herein). DCFH-DA is cleaved by cellular esterases upon entry into the cells, trapping the nonfluorescent 2′,7′-dichlorofluorescin (DCFH) inside. ROS, particularly hydrogen peroxide (H₂O₂) and hydroxyl radical generate the fluorescent product dichlorofluorescein (DCF) by causing the oxidation of DCFH. Increases in DCF fluorescence result from DCFH oxidation to DCF. DCF fluorescence was measured at an excitation wavelength of 480 nm, and a 520 nm band pass emission filter. Attention was focused on the maximal DCF fluorescence intensity value during the first minutes of reperfusion for comparison. All measurements were expressed in arbitrary units (a.u.) of fluorescence.

Data Analysis

For each experiment a field of about 500 cells was observed. Treatment and control groups were matched in sets containing cells isolated and cultured on the same day so as to eliminate variability due to cell batch. Additional 25 mm coverslips were used for replicate experiments (“n”). Results are reported as means plus or minus S.E.M. and two-tailed unpaired t-tests comparing similar time points throughout ischemia and reperfusion were used as tests of significance, with p<0.05 considered to be significant.

### EXAMPLE 2

Materials

Propidium iodide and 2′7′-dichlorofluorescin diacetate were obtained from Molecular Probes (Eugene, Oreg.); 4,5-diaminofluorescin diacetate (DAF-2 DA) was obtained from EMB Biosciences (San Diego, Calif.). N^{G}-nitro-L-arginine methyl ester (L-NAME), apocynin and stigmatellin were obtained from Sigma Chemical (St. Louis, Mo.).

Cardiac Cell Culture

Ventricular embryonic chick cardiomyocytes were prepared according to published procedure. (Vanden Hoek T L, Becker L B, Shao Z, et al: Reactive oxygen species released from mitochondria during brief hypoxia induce preconditioning in cardiomyocytes, J. Biol. Chem. 199; 273:18092-18098, which is incorporated herein by reference). 10-day old chicken embryo hearts were removed, ventricular tissue minced into 0.5 mm fragments, enzymatically dispersed with 0.25% trypsin (Life Technologies, New York, N.Y.), centrifuged, 0.7×10⁶ cells were pipetted onto 25 mm coverslips, and incubated with 5% CO₂. Coverslips were checked for non-muscle cell contamination. Experiments were performed with 3-5 day cardiac cell cultures, by which time a synchronously contracting layer of cells could be visualized and viability exceeded 95%.

Perfusion

Coverslips with contracting cells were placed in a 1.2 mL Sykes-Moore perfusion chamber (Bellco Glass Inc., Vineland, N.J.). The chamber and inflow tubing were maintained at 37° C. Perfusate was supplied to the chamber (0.25 ml/min) via stainless steel tubing to minimize diffusive entry of ambient O₂.

To simulate ischemia, the perfusate contained BSS with 2-deoxyglucose (2-DOG) (20 mM) rather than glucose, 18 mM NaCO₃ and [K]⁺ of 8.0 mEq/L, and was equilibrated with 80% N₂ and 20% CO₂ to produce a final PO₂ of 3-5 torr, PCO₂ of 144 torr, and pH 6.8. In addition, 20 mM of glycolytic inhibition 2-deoxyglucose was added to better model adenosine triphosphate depletion.

For perfusion, three perfusate solutions were used: normocarbic reperfusion perfusate, hypocarbic reperfusion perfusate and hypercarbic reperfusion perfusate. Normocarbic perfusate used for baseline conditions and for reperfusion subsequent to ischemia contained oxygenated balanced salt solution (BSS) with a PO₂ OF 149 torr, PCO₂ of 36 torr, pH of 7.4, and [K]⁺ of 4.0 mEq/L, 18 mM NaHCO₃ and 5.6 mM glucose. Hypocarbic perfusate consisted of standard BSS equilibrated with 78% N₂, 1% CO₂ , and 21% O₂ to achieve a PO2 of 149 torr, Pco₂ of 7 torr and a pH of 7.9. Hypercarbic perfusate consisted of BSS with 10.7 mM NaHCO₃ that was equilibrated with 69% N₂, 10% CO₂, and 21% oxygen to yield a PO₂ of 149 torr, Pco₂ of 71 torr and a pH of 6.8. Cells were exposed to 1 hr of simulated ischemia and 3 hours of reperfusion (normocarbic, hypercarbic, and hypercarbic).

Light and Fluorescence Microscopy

Cells were imaged using an inverted phase/epifluorescent microscope (Nikon, Diaphot 300, Japan) with a mercury light source (Nikon, 75W, Japan) and a cooled digital camera (Photometrics, Cools-SNAP, Japan). The microscope was also equipped with phase contrast objective (Nikon, ph1 DLL, Japan) to illuminate cells. Fluorescent cell images were obtained using a ×10 or ×20 objective (Nikon, DIC, Japan). Data was acquired and analyzed with the Metafluor software (Universal Imaging).

Viability Assay

Cell viability was assessed with the fluorochrome propidium iodide (PI) (5 μM, Molecular Probes, Eugene, Oreg.). PI is excluded from viable cells, but enters cells and binds to chromatin after loss of cell membrane integrity. The cells then become highly fluorescent (excitation wavelength of 540 nm and a 590 nm band pass emission filter). PI was used to quantify cell death throughout the entire time of each experiment. PI exhibited minimal toxicity in control cells even after a ten-hour exposure. All cells in the field studied were stained with PI at the end of the experiment by permeabilizing the cells with digitonin (300 mM). Percent cell death was then calculated as the PI fluorescence at any given time point relative to the maximal fluorescence value seen after digitonin exposure.

Cell Contraction

Contractions were assessed by monitoring synchronous movement within the same field of cells as previously reported. A return of contraction following simulated ischemia/reperfusion was indicated when contractions could be seen throughout the field of cells following the three hour period of reperfusion.

Measurement of Intracellular Reactive Oxygen Species (ROS)

Intracellular oxidant stress due to ROS was monitored real time with the intracellular probe 2′,7′-dichlorofluorescin diacetate (DCFH-DA, 5 mM, Molecular Probes, Eugene, Oreg.) as described in published literature (Vanden Hoek, F L, et al., Reactive oxygen species released from mitochondria during brief hypoxia induce preconditioning in cardiomyocytes, J. Biol. Chem. 1988; 273:18092-18098, which is incorporated by reference herein; Vanden Hoek, T L, et al, Preconditioning in cardiomyocytes protects by attenuating oxidant stress at reperfusion, Circ Res 2000; 86:534-540, which is incorporated by reference herein; Vanden Hoek, T L, Reperfusion, not simulated ischemia initiates intrinsic apoptosis injury in chick cardiomyocytes Am J. Physiol Heart Physiol 2003; 284:H141-150, which is incorporated by reference herein). DCFH-DA is cleaved by cellular esterases upon entry into the cells, trapping the nonfluorescent 2′,7′-dichlorofluorescin (DCFH) inside. ROS, particularly hydrogen peroxide (H₂O₂) and hydroxyl radical generate the fluorescent product dichlorofluorescein (DCF) by causing the oxidation of DCFH. Increases in DCF fluorescence result from DCFH oxidation to DCF. DCF fluorescence was measured at an excitation wavelength of 480 nm, and a 520 nm band pass emission filter. Attention was focused on the maximal DCF fluorescence intensity value during the first minutes of reperfusion for comparison. All measurements were expressed in arbitrary units (a.u.) of fluorescence.

Data Analysis

For each experiment a field of about 500 cells was observed. Treatment and control groups were matched in sets containing cells isolated and cultured on the same day so as to eliminate variability due to cell batch. Additional 25 mm coverslips were used for replicate experiments (“n”). Results are reported as means plus or minus S.E.M. and two-tailed unpaired t-tests comparing similar time points throughout ischemia and reperfusion were used as tests of significance, with p<0.05 considered to be significant. Cell death and fluorescence data were analyzed by two-way repeated analysis of variance with one between-group factor (type of reperfusion) and one repeated-measures factor (time). Both the group main effect and the group-by-time interaction were tested for significance, and the latter incorporated a Greenhouse-Geisser correction to the degrees of freedom to allow for lack of sphericity (i.e., unequal variances and serial correlation over time). If either main group effect or the group-by-time interaction term was statistically significant, subsequence comparisons were performed at each time point using a two-sample Student's t-test allowing for unequal variances. Cell contraction data was analyzed by Fisher's exact test. We consider p<0.05 to be statistically significant.

### EXAMPLE 3

Inhibition of Superoxide Dismutase by Diethyldithiocarbamic Acid (DDC)

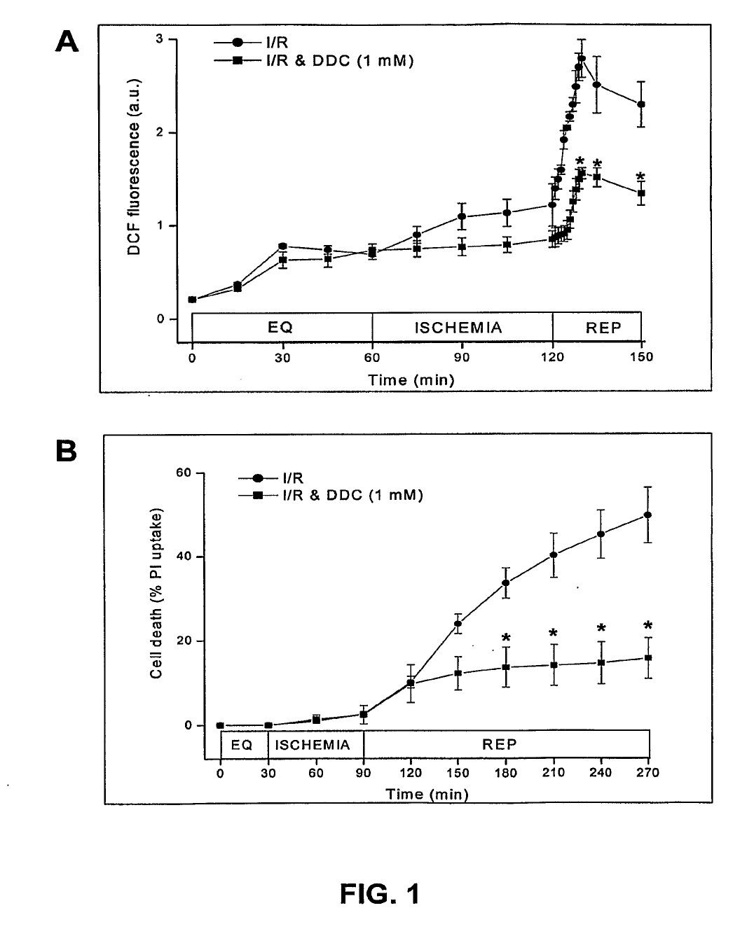

Cells prepared according to Example 1 were contacted with 1 mM DDC during simulated ischemia and the first 15-30 minutes of reperfusion. As shown in FIG. 1A, DDC (1 mM) reduced maximal DCF intensity from a control of 2.8±0.2 a.u. to 1.6±0.1 a.u. (n=5, p<0.01). As shown in FIG. 1B, DDC (1 mM) decreased cell death from 49.7±6.7% to 15.7±4.7% (n=5, p<0.01).

### EXAMPLE 4

Inhibition of NADH Oxidase With 2-Anthracene-Carboxylic Acid

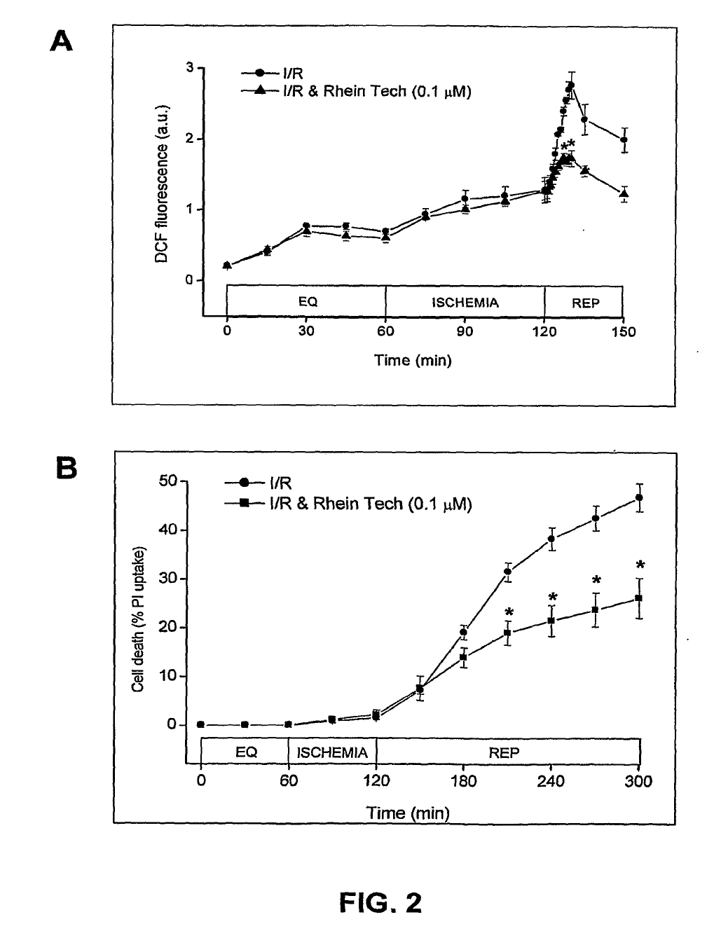

Cells prepared according to Example 1 were contacted with 0.1 μM 2-anthracene-carboxylic acid during the first 10 minutes of reperfusion. As shown in FIG. 2A, 2-anthracene-carboxylic acid (0.1 μM) reduced maximal DCF intensity from a control of 2.8±0.2 a.u. to 1.7±0.1 a.u. (n=3, p<0.05). As shown in FIG. 2B, 2-anthracene-carboxylic acid (1 μM) decreased cell death from 47.1±3.0% to 26.1±4.0% (n=5, p<0.01). Synchronous contractions returned in all treated groups (5/5) compared to no return of contractions in control cells (0/7).

### EXAMPLE 5

Competitive Inhibition of β-NADH With α-NADH

Cells prepared according to Example 1 were contacted with 20 μM of exogenous β-NADH during the first 10 minutes of reperfusion. The percent increase in ROS generation within the first 10 minutes of reperfusion was increased over ischemia levels for cells treated with 20 μM of exogenous β-NADH. ROS increased from 86.2±19.6% in control cells to 511±123% (n=8, p<0.01) in cells contacted with 20 μM of exogenous β-NADH. Concomitant addition of the inactive α-NADH, which competitively inhibits the physiologically active beta form, blocked this increase in ROS.

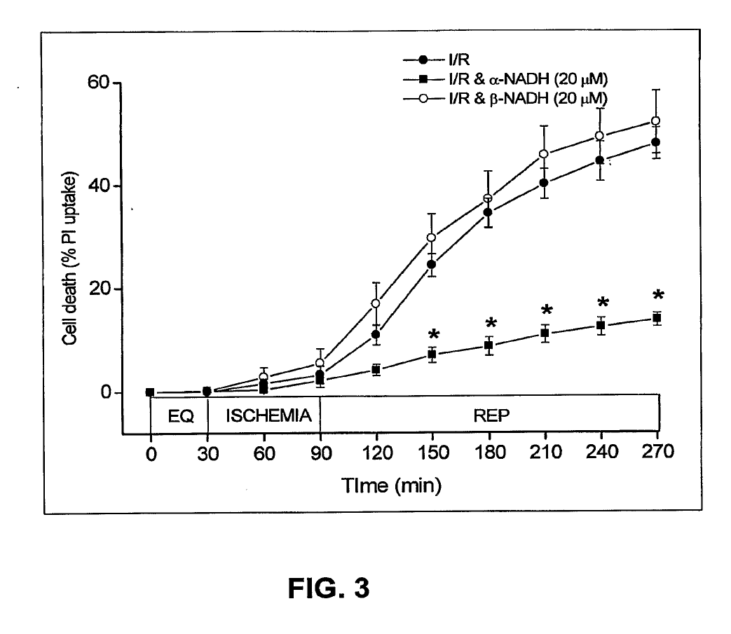

As shown in FIG. 3, α-NADH (20 μM) decreased cell death from 47.1±3.0% to 13.8±1.3% (n=5, p<0.01). Cells treated with exogenous β-NADH (20 μM) had similar levels of cell death as compared to controls. Contractions returned in all α-NADH treated groups (5/5), compared to the absence of contractile return in the β-NADH treated cells or control cells.

### EXAMPLE 6

Inhibition of Complex III With Stigmatellin

Cells prepared according to Example 1 were contacted with stigmatellin (2-20 nM) during the first 15 minutes of reperfusion. As shown in FIG. 4A, stigmatellin (2-20 nM) attenuated peak DCF fluorescence intensity from 2.4±0.3 a.u. to 0.46±0.02 a.u. (n=5, p<0.001). As shown in FIG. 4B, stigmatellin (2-20 nM) decreased cell death from 53.8±3.5% to 10.8±2.9% (n=5, p<0.001). Contractions returned in all stigmatellin treated groups (5/5), compared to the absence of contractile return in the control cells.

### EXAMPLE 7

Inhibition of Complex I Via Rotenone

Cells prepared according to Example 1 were contacted with rotenone (10 μM, 1 μM and 100 nM) during the first 15 minutes of reperfusion. As shown in FIG. 5A, rotenone (10 μM, 1 μM and 100 nM) increased cell death from a control of 50.2±3.0% to 66.1±3.4% (n=3, p<0.01), 77.4±6.6% (n=3, p<0.01) and 61.3±1.6% (n=3, p<0.01), respectively. As shown in FIG. 5B, rotenone is able to attenuate reperfusion ROS (dose is 5 μM, but is representative of all doses) from a peak fluorescence intensity of 2.4±0.3 a.u. to 0.7±0.1 a.u. (n=5, p<0.01).

### EXAMPLE 8

Effects of Hypercarbic and Hypocarbic Conditions on Cell Viability

Cells were prepared as described in Example 2, where cells were equilibrated with normoxic balanced salt solution for 30 minutes, and subjected to simulated ischemia for 1 hour and reperfusion with adjusted CO₂ levels for 3 hours and cell viability monitored. As seen in FIG. 7 cell viability during ischemia before normocarbic, hypocarbic or hypercarbic conditions had no significant difference. Following ischemia at 270 minutes, cell death in hypercarbic reperfusion was significantly higher (80.4±4.5%, n=5) compared with normocarbic reperfusion (54.8%±4.0%, n=10, p<0.01), while hypercarbic reperfusion resulted in significantly lower cell death (26.3±2.8%, n=15, p<0.001) as compared to normocarbic perfusion, FIG. 7. All groups demonstrated some component of post-resuscitation, (i.e., reperfusion) injury which was significantly modulated by altering the decrease if tissue CO₂ induced at reperfusion, as demonstrated by the return of spontaneous cell contraction, which was seen in 0 of 5 of the hypocarbic groups, 3 of 10 in the normocarbic group (p=0.5) and 10 of 15 in the hypercarbic groups (p=0.033).

### EXAMPLE 9

Effects of Hypocarbic or Hypercarbic Conditions on Oxidant Generation

The affect of hypocarbic and hypercarbic reperfusion on the generation of reactive oxygen species (ROS) was tested. Cells were prepared as described in Example 2, and the level of dichlorofluorescin (DCF) fluorescence, an indicator of ROS generation, during conditions of hypocarbic, normocarbic, and hypercarbic reperfusion was assayed. As seen in FIG. 7A, hypocarbic reperfusion resulted in an increase of the oxidative burst at 96 minutes, 6.0±1.0 arbitrary units (au), n=6 vs. 3.4±0.4 au in the normocarbic group, n=4 while hypercarbic reperfusion decreased the maximal DCF fluorescence from 3.4±0.4 au to 1.8±0.1 au (n=4, p<0.05).

### EXAMPLE 10

Inhibition of the Oxidant Generation by NOS Inhibitor

To test if the decrease in reperfusion ROS associates with hypercarbic conditions was also related to NO generation, the ability of a NOS inhibitor, L-NAME to inhibit ROS generation was tested. Cells were prepared as described in Example 2, and treated with L-NAME (200 μM) as described previously (16) during a 2-hour pre-incubation period as well as equilibration and I/R (n=5). As seen in FIG. 7B, L-NAME reversed the decreased DCF fluorescence induced by hypercarbic reperfusion, from 1.7±1.0 au to 3.8±0.7 au at 98 minutes (p<0.05).

### EXAMPLE 11

Effects of Hypocarbic and Hypercarbic Conditions on NO Generation

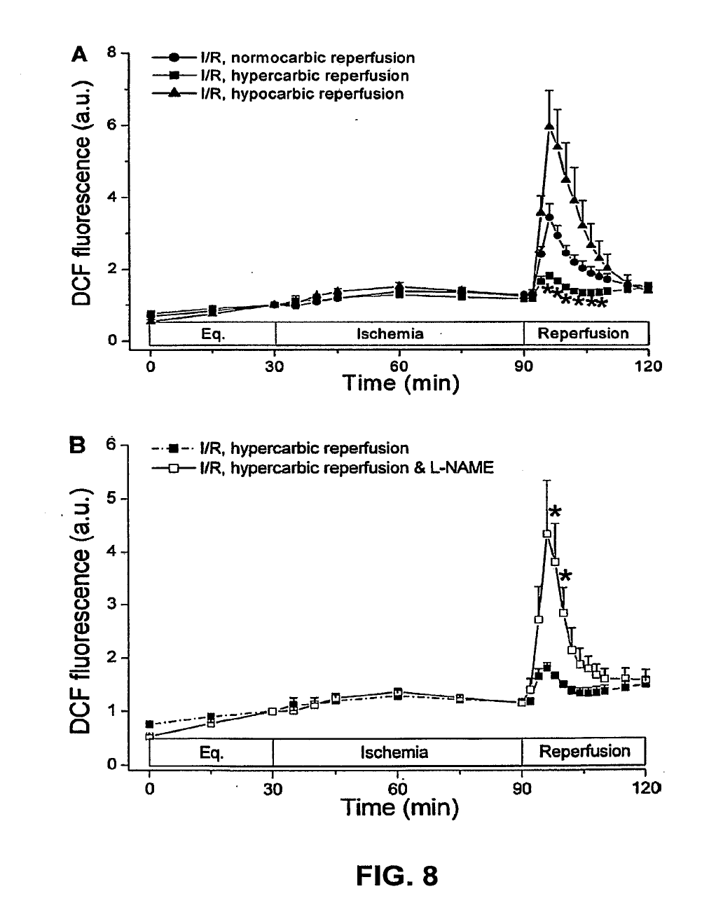

Endogenous NO generation has been implicated to have both protective and detrimental roles in various in vitro and in vivo models of I/R injury. To test whether endogenous NO was affected by hypocarbic or hypercarbic reperfusion, we continuously monitored the entire course of I/R using the NO indicator DAF-2 DA of cells treated as described in Example 2. As shown in FIG. 8A, the NO generation was greater in the hypocarbic condition group during the first minutes of reperfusion, resulting in a significantly higher peak level of NO at 15 minutes of reperfusion (n=8, p<0.05) compared with the normocarbic control, wherein the NO level declined gradually as did the levels of the normocarbic group, exhibiting no different at 1 hour of reperfusion. In contrast, as seen in FIG. 8B, no significant differences in DAF-2 fluorescence was seen between hypercarbic and normocarbic condition groups, although after the 15 minute peak in NO levels, the hypercarbic reperfusion resulted in sustained generation of NO compared to the gradual decline on normocarbic reperfusion, leading to a significantly higher level of NO in the later phase of reperfusion (n=8, p<0.01).

### EXAMPLE 12

L-NAME Inhibits Sustained NO Levels Induced by Hypercarbic Reperfusion

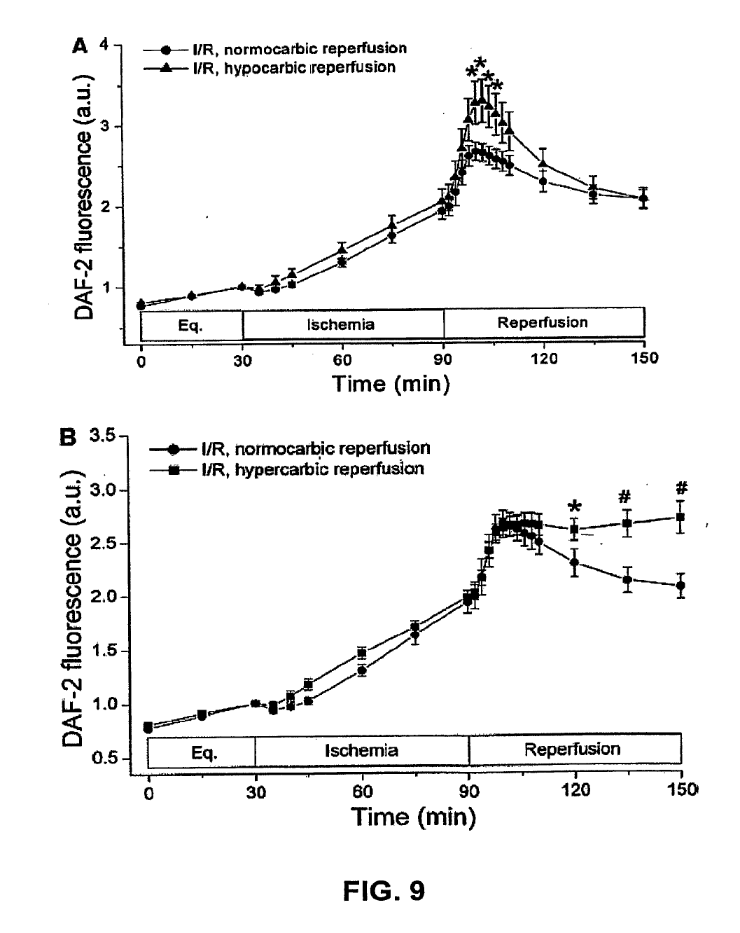

Cells were treated as described in Example 11, and the NO profile and cell viability was assessed in hypercarbic reperfusion compared with hypercarbic reperfusion with 200 μM L-NAME incubated during I/R as described above. As seen in FIG. 9A, co-treatment of L-NAME blocked maintenance of the sustained NO levels induced by hypercarbic reperfusion (n=8, p<0.05) and reversed the protection of hypercarbic on cell death by increasing cell death from 28.3%±3.2% to 54.3%±6.0 at 270 minutes, n=3 in each group, p<0.05.

### EXAMPLE 13

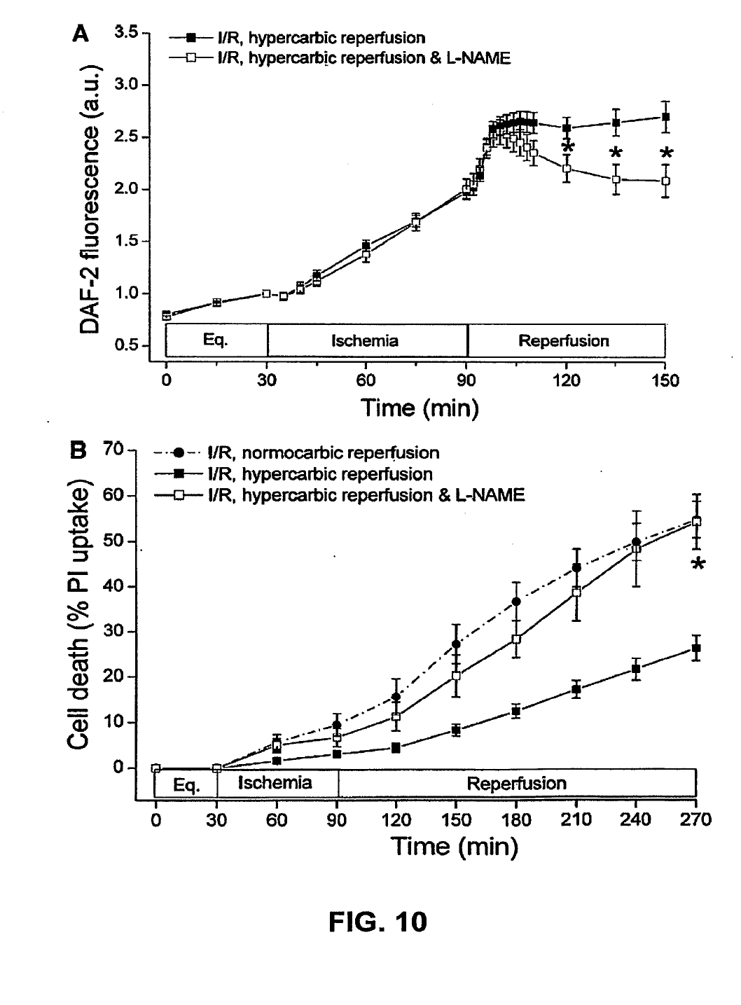

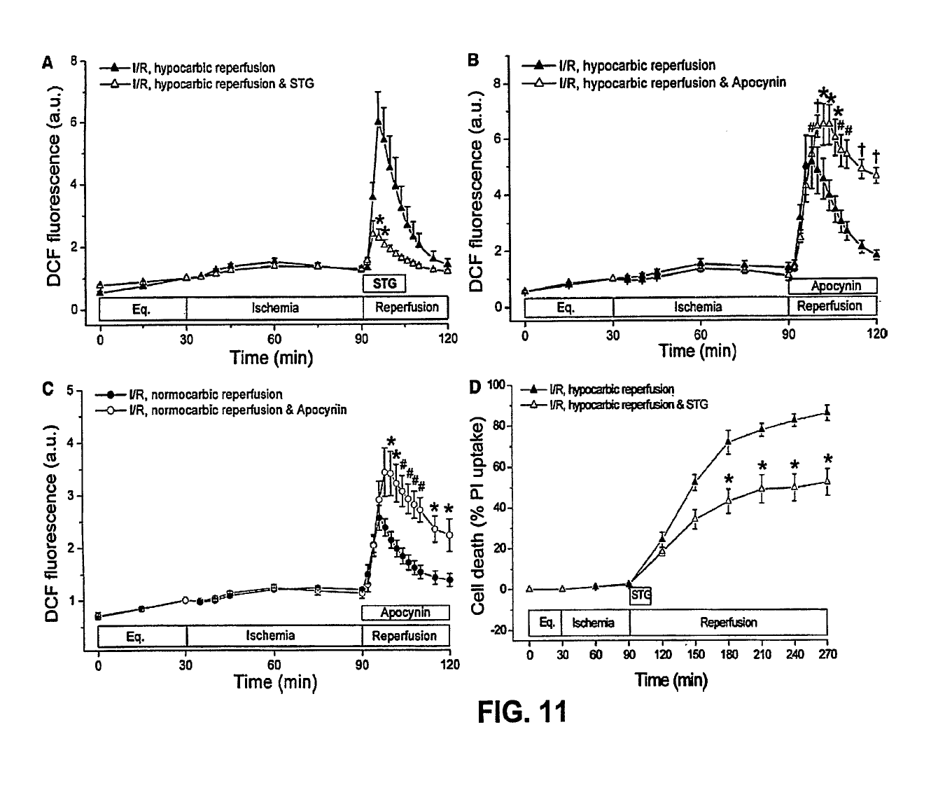

Stigmatellin blocks the increased reperfusion ROS and reduces cell death caused by hypocarbic reperfusion. To test if mitochondria are a major source of reperfusion oxidant burst induced by hypocarbic conditions, then inhibition of the respiratory chain should also attenuate the burst of DCF fluorescence seen at hypocarbic reperfusion. Cells were treated as described in Example 2. As shown in FIG. 10A, the addition of stigmatellin (20 nM) given during the first 15 minutes of reperfusion only of cells treated as described in example 1 significantly attenuated the burst of DCF fluorescence seen during hypocarbic reperfusion from 2.3±0.3 au to 6.0±1.0 au, n=5 in each group, p<0.05. IN contrast, blockade of cytoplasmic membrane nicotinamide adenine dinucleotide phosphate (reduced) (NADPH) oxidase with apocynin (300 μM) did not attenuate the reperfusion ROS burst, either during hypocarbic (FIG. 10B) or normocarbic (FIG. 10C) reperfusion.

As seen in FIG. 10D, stigmatellin treatment attenuated the increase of cell death caused by hypocarbic perfusion at 270 minutes, from 85.9%±4.5% (hypocarbic perfusion alone) to 52.2%±6.5% (n=5 in each group, p<0.01).

1. A method of reducing cell death in a population of cells following hypoxia comprising reoxygenating the cells in the presence of an effective amount of a reversible electron transport chain inhibitor.

2. (canceled)

5. The method of claim 1, wherein the reversible electron transport chain inhibitor is a competitive inhibitor.

6. The method of claim 1, wherein the electron transport chain inhibitor is a Complex III inhibitor.

7. The method of claim 6, wherein the Complex III inhibitor is stigmatellin.

8. The method of claim 7, wherein the cells are contacted with stigmatellin at a concentration of about 2 nM to about 20 nM.

9. The method of claim 7, wherein the stigmatellin is used during about the first 15 minutes of reoxygenation.

10. The method of claim 1, wherein the electron transport chain inhibitor is a NADH-linked enzyme inhibitor.

11. The method of claim 10, wherein the NADH-linked enzyme is NADH CoQ oxidoreductase.

12. The method of claim 10, wherein the NADH-linked enzyme inhibitor is 2-anthracene-carboxylic acid.

13. The method of claim 12, wherein the 2-anthracene-carboxylic acid is used in a concentration of about 0.1 μM.

14. The method of claim 12, wherein the 2-anthracene-carboxylic acid is used during about the first 10 minutes of reoxygenation.

15. The method of claim 1, wherein the electron transport chain inhibitor is an NADH-linked enzyme antagonist.

16. The method of claim 15, wherein the NADH-linked enzyme antagonist is α-NADH.

17. The method of claim 15, wherein the α-NADH is used in a concentration of about 20 μM.

18. The method of claim 15, wherein the α-NADH is used during about the first 15 minutes of reoxygenation.

19. The method of claim 1, wherein the electron transport chain inhibitor is a copper chelating agent.

20. The method of claim 19, wherein the copper chelating agent is diethyldithiocarbamate.

21. The method of claim 20, wherein the diethyldithiocarbamate is used in a concentration of about 1 mM.

22. The method of claim 20, wherein the diethyldithiocarbamate is used during about the first 15 to about 30 minutes of reoxygenation.

23. The method of claim 1, wherein the cells are in a subject.

24. The method of claim 23, wherein contacting the cells comprises administering an effective amount of reversible electron transport chain inhibitor to the subject.

25. The method of claim 1, wherein the population of cells are comprised within a harvested organ or tissue-c.

26. A method of determining the effectiveness of a reversible electron transport chain inhibitor for reducing cell death in a population of cells following hypoxia comprising
(a) reoxygenating the cells in the presence of a reversible electron transport chain inhibitor; and
(b) assessing the effect on cell death.

27. A method of reducing cell death in a population of cells following hypoxia comprising reoxygenating the cells with oxygen under a hypercarbic condition.

28-30. (canceled)

31. The method of claim 24, wherein the population of cells is comprised within a harvested organ or tissue, wherein the hypercarbic condition includes a pCO₂ greater than a normocarbic condition.

32. The method of claim 27, wherein the pCO₂ is greater than 40 torr.

33. The method of claim 32, wherein the pCO₂ is about 70 torr.

34. The method of claim 27, wherein the hypercarbic condition is administered during about the first 15 minutes of reoxygenation.

35. The method of claim 27, wherein the hypercarbic condition is administered during about the first 15 to about 30 minutes of reoxygenation.

36. The method of claim 27, further comprising contacting the cells with an effective amount of a reversible electron transport chain inhibitor.
